(19) United States
(12) Patent Application Publication
(10) Pub. No.: US 2023/0220476 A1
(43) Pub. Date: Jul. 13, 2023

(54) METHOD OF DIAGNOSING MITOCHONDRIAL DNA DISORDERS USING STOOL SAMPLES

(71) Applicant: University of Newcastle Upon Tyne, Newcastle Upon Tyne, Tyne and Wear (GB)

(72) Inventors: Gráinne GORMAN, Newcastle upon Tyne (GB); David HOUGHTON, Newcastle upon Tyne (GB); Laura GREAVES, Newcastle upon Tyne (GB)

(21) Appl. No.: 18/009,625

(22) PCT Filed: Jun. 9, 2021
(86) PCT No.: PCT/GB2021/051425

§ 371 Date: Dec. 9, 2022

(30) Foreign Application Priority Data
Jun. 10, 2020 (GB) 2008787.0

(51) Int. Cl. C12Q 1/6883; A61K 31/122; A61K 31/385; A61K 31/195; A61K 31/197; A61K 45/06

(52) U.S. Cl. CPC C12Q 1/6883; A61K 31/122; A61K 31/385; A61K 31/195; A61K 31/197; A61K 45/06; C12Q 2600/156; C12Q 2600/118

(57) ABSTRACT

A method for providing a diagnosis and/or prognosis for a mitochondrial DNA (mtDNA) disorder or determining the risk of a mtDNA disorder developing in a subject, the method comprising the steps of assaying a stool sample from the subject to measure the level of mutated mtDNA molecules is provided. Methods for monitoring the progression of a mtDNA disorder, evaluating therapeutic effect of a treatment for a mtDNA disorder, and determining a subjects compliance with a prescribed treatment for a mtDNA disorder using a stool sample are also provided. Further, methods of treating a mtDNA disorder and uses of a stool sample in the methods described herein are also provided.

### FIELD OF THE INVENTION

The present invention relates to a method for providing a diagnosis and/or prognosis for a mitochondrial DNA (mtDNA) disorder or for determining the risk of a mtDNA disorder developing in a subject. The invention also relates to a method for evaluating a therapeutic effect of a treatment for a mtDNA disorder, a method for determining a subject's compliance with a prescribed treatment for a mtDNA disorder, and a method for treating a mtDNA disorder. The invention also relates to a method for evaluating the severity of a mtDNA disorder. Further, the invention relates to the use of a stool sample in the methods described herein.

### INTRODUCTION

Mitochondrial diseases are a group of genetic disorders that are highly heterogeneous, with substantial variability in disease burden and progression, although almost every organ in body can be affected. The cause for the vast heterogeneity of mitochondrial diseases is two-fold.

Firstly, mitochondrial diseases can be caused by mutations in hundreds of different genes in nuclear DNA (nDNA) or mtDNA. Secondly, mutations in mtDNA are further complicated by mtDNA heteroplasmy (also known as mutation load), the level of which is generally associated with the age of onset of the disease and/or disease severity.

For this reason, in the context of mitochondrial diseases caused by mtDNA mutations, assessing levels of mtDNA heteroplasmy is of important diagnostic and prognostic value. However, the problem with measuring the level of mtDNA heteroplasmy is that the levels have been found to be tissue-specific. This means that not all tissues can be used to accurately measure heteroplasmy in a clinical and research setting.

Current methods for accurately detecting and quantitatively measuring the levels of heteroplasmy include measuring the levels in a skeletal muscle biopsy (recognised as the gold standard), blood or a urine sample. Skeletal muscle biopsies are the most clinically useful tissue to understand mtDNA heteroplasmy and accurately diagnose disease status and disease burden. However, muscle biopsy is an invasive, a costly procedure and most clinical and research teams do not have the capability to routinely take and analyse skeletal muscle biopsies. Additionally, in babies suspected to have mitochondrial diseases, obtaining a skeletal muscle biopsy is dangerous. Therefore, muscle biopsy is generally not the primary diagnostic course of action for suspected mtDNA disease. Blood and urine samples are more commonly used due to costs and ease of access; however, these have been shown to be less accurate than skeletal muscle, due to age and gender specific factors. Furthermore, blood and urine are also notoriously difficult to obtain in babies.

Accordingly, an improved method for measuring mtDNA heteroplasmy for diagnostic purposes is needed.

### SUMMARY OF THE INVENTION

The present invention is based on the inventors' surprising finding that stool samples have a mtDNA heteroplasmy level that highly correlates to the levels in muscle tissue. This correlation is unexpected given the heteroplasmy mismatch between the muscle and many other tissues, such as blood, urine or even skin.

The inventors have tested stool samples obtained from 17 patients diagnosed with a mtDNA disorder to determine the heteroplasmy levels and compared them to the heteroplasmy levels determined in a muscle biopsy. Unexpectedly, the inventors found the levels of heteroplasmy to be substantially the same as those found in the muscle biopsy, which has allowed the inventors to develop a new method for diagnosing mtDNA diseases. This new method is highly advantageous for a number of reasons, such as ease of obtaining stool samples and reduced assay cost.

Accordingly, in one aspect the present invention relates to a method for providing a diagnosis and/or prognosis for a mtDNA disorder or for determining the risk of a mtDNA disorder developing in a subject, the method comprising the steps of:
a) assaying a stool sample from the subject to measure the level of mutated mtDNA molecules; and
b) comparing the level to a reference value, wherein an increase in the level as compared to the reference value is indicative of the mtDNA disorder or of an increased risk of the mtDNA disorder developing in a subject.

Suitably, the reference value may be the level of mutated mtDNA molecules in a sample from a healthy subject.

Suitably, the healthy subject might not have a mtDNA disorder or is not at risk of developing a mtDNA disorder.

Suitably, the sample from the healthy subject may be selected from the group consisting of a stool sample, a muscle biopsy sample, a blood sample, and a urine sample.

In one aspect the invention relates to a method for evaluating therapeutic effect of a treatment for a mtDNA disorder, the method comprising the steps of:
a) assaying a stool sample from a subject before treatment to measure the level of mutated mtDNA molecules;
b) assaying a stool sample from the subject after treatment to measure the level of mutated mtDNA molecules; and
c) comparing the level obtained in step a) and step b), wherein a decrease in the level obtained in step b) as compared to the level in step a) is indicative of the treatment having therapeutic effect.

In one aspect the invention relates to a method for determining a subject's compliance with a prescribed treatment for a mtDNA disorder, the method comprising:
a) assaying a stool sample from the subject before treatment to measure the level of mutated mtDNA molecules;
b) assaying a stool sample from the subject after treatment to measure the level of mutated mtDNA molecules; and
c) comparing the level obtained in step a) and step b), wherein a decrease in the level obtained in step b) as compared to the level in step a) indicates that the subject is/and or has complied with the prescribed treatment.

In one aspect, the invention relates to a method for monitoring the progression of a mtDNA disorder, the method comprising the steps of:
a) assaying a stool sample from the subject to measure the level of mutated mtDNA molecules;
b) repeating step a) for the same subject after a time interval; and
c) comparing the level obtained in step a) and step b), wherein a change in the levels identified in a) and b) indicates a change in the progression of the mtDNA disorder in the subject.

Suitably, in the context of the methods of the invention, assaying may comprise extracting the mtDNA molecules from the sample.

Suitably, in the context of the methods of the invention, assaying may comprise amplifying mtDNA molecules or fragments thereof.

Suitably, in the context of the methods of the invention, amplifying may be by a method selected from the group consisting of polymerase chain reaction, loop mediated isothermal amplification, nucleic acid sequence based amplification, strand displacement amplification, rolling circle amplification and ligase chain reaction.

Suitably, in the context of the methods of the invention, the method may further comprise sequencing mtDNA molecules or fragments thereof.

Suitably, in the context of the methods of the invention, sequencing may be by pyrosequencing, whole genome sequencing, and/or Sanger sequencing

Suitably, in the context of the methods of the invention, the level of mutated mtDNA molecules may be determined by the proportion of mutated mtDNA molecules to non-mutated mtDNA molecules in the sample, the percentage of mutated mtDNA molecules in the sample, or the number of mutated mtDNA molecules in the sample.

Suitably, in the context of the methods of the invention, the subject may be a mammal (for example a human, a mouse, a rat, a monkey, a horse, a dog, or a cat).

Suitably, in the context of the methods of the invention, the subject may be a child.

Suitably, in the context of the methods of the invention, the mtDNA disorder may be selected from the group consisting of mitochondrial encephalopathy with lactic acidosis and stroke-like episodes (MELAS); chronic progressive external ophthalmoplegia (CPEO); myoclonic epilepsy with ragged-red fibers (MERRF); Leber's hereditary optic neuropathy (LHON), Leigh syndrome; Kearns-Sayre syndrome (KSS); neuropathy, ataxia, retinitis pigmentosa (NARP); Alpers-Huttenlocher syndrome; ataxia neuropathy syndromes (ANS); Pearson's syndrome; infantile myopathy and lactic acidosis (fatal and non-fatal forms); and progressive brain-stem disorder (MILS).

Suitably, in the context of the methods of the invention, the mutated mtDNA molecule may comprise a mutation in a gene and/or in a non-coding region of the mtDNA.

Suitably, in the context of the methods of the invention, the gene may encode a tRNA, a protein and/or a rRNA

Suitably, in the context of the methods of the invention, the gene encoding a tRNA may be MT-TL1.

Suitably, in the context of the methods of the invention, the mutation may be m.3243A>G.

Suitably, in the context of certain methods of the invention, the treatment may be selected from the group consisting of coenzyme Q10, B complex vitamins, alpha lipoic acid, L-carnitine, creatine, L-Arginine, endurance exercise, and resistance training.

In a further aspect, the present invention relates to a method of providing a diagnosis and/or prognosis for a mtDNA disorder or for determining the risk of a mtDNA disorder developing in a subject, and treating or preventing the mtDNA disorder, the method comprising the steps of:
a) assaying a stool sample from the subject to measure the level of mutated mtDNA molecules;
b) comparing the level to a reference value, wherein an increase in the level as compared to the reference value is indicative of the mtDNA disorder or of an increased risk of the mtDNA disorder developing in a subject; and
c) administering the subject that has been identified as having or being at risk of having mtDNA disorder a treatment for mtDNA disorder, thereby treating the subject.

In another aspect, the present invention provides a method of treating a mtDNA disorder in a subject, the method comprising:
a) requesting a test providing the results of an analysis to determine the level of mutated mtDNA molecules in a stool sample from the subject and comparing the level to a reference value, wherein an increase in the level as compared to the reference value is indicative of the mtDNA disorder or of an increased risk of the mtDNA disorder developing in a subject; and
b) administering the subject that has been identified as having or being at risk of having mtDNA disorder a treatment for mtDNA disorder, thereby treating the subject.

In a further aspect, the present relates to the use of a stool sample in a method as descried herein.

It will be appreciated that, except for where the context requires otherwise, the considerations set out in this disclosure should be considered to be applicable to all aspects of the invention.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 depicts a picture of an agarose gel. The bands correspond to a 21-base pair mtDNA fragment that was extracted from a stool sample and amplified by PCR for the 3243 A>G mutation.

FIG. 2 is a graph showing levels of mtDNA heteroplasmy in stool sample 1 (n=17) and stool sample 2 (n=17).

FIG. 3 is a graph comparing heteroplasmy in a muscle sample, stool sample, and blood sample.

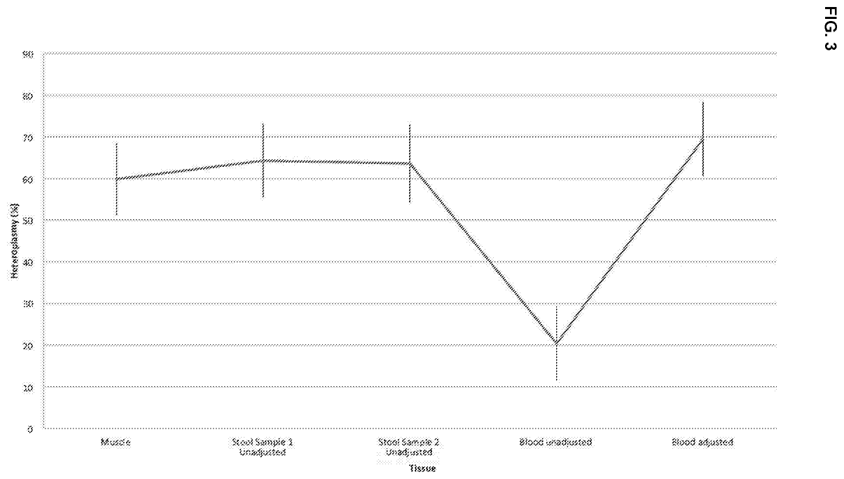

### DETAILED DESCRIPTION OF THE INVENTION

The invention provides a new and surprising approach for diagnosis and/or prognosis of a mtDNA disorders, determining the risk of a mtDNA disorder developing in a subject, evaluating a therapeutic effect of a treatment for mtDNA disorder, monitoring a subject's compliance with a treatment for a mtDNA disorder and/or evaluating the severity of a mtDNA disorder.

This new approach is based on the inventors' unexpected finding that stool samples are reliable for the purpose of determining mtDNA heteroplasmy. This reliability is due to the fact that stool has substantially the same level of mtDNA heteroplasmy as in skeletal muscle tissue. Currently, measuring heteroplasmy in the muscle through obtaining a muscle biopsy is the gold standard method by which mtDNA heteroplasmy is determined.

Methods for Diagnosing a mtDNA Disorder or Determining the Risk of a mtDNA Disorder Developing in a Subject

In one aspect, the invention relates to a method for providing diagnosis and/or prognosis for a mtDNA disorder or for determining the risk of a mtDNA disorder developing in a subject, the method comprising the steps of:
a) assaying a stool sample from the subject to measure the level of mutated mtDNA molecules; and
b) comparing the level to a reference value, wherein an increase in the level as compared to the reference value is indicative of the mtDNA disorder or of an increased risk of the mtDNA disorder developing in the subject.

Suitably, the method may be an in vitro method for providing a diagnosis or prognosis for a mtDNA disorder or determining the risk of a mtDNA disorder developing in a subject.

The term “subject” as used herein refers to any individual diagnosed with a mtDNA disorder, suspected of having a mtDNA disorder, or believed to be at risk of a mtDNA disorder. Suitably, the subject may be a mammal, such as a human, mouse, rat, monkey, horse, dog, or cat. More suitably the subject is a human (for example a child). The methods described herein are particularly advantageous in the context of young children (for example from 0 to about 3 years of age) in which obtaining a muscle biopsy may be dangerous. In the context of the present specification the terms “subject”, “individual”, and “patient” are used herein interchangeably.

The subject may be symptomatic (e.g., the subject presents symptoms associated with a mtDNA disorder), or the subject may be asymptomatic (e.g., the subject does not present symptoms associated a mtDNA disorder). Whether the subject is symptomatic or asymptomatic may depend, for example, on the location of a mutation in mtDNA, mutation load (level of mutated mtDNA molecules), and/or the subject's age.

The term “mtDNA disorder” as used herein refers to a pathological condition wherein the level of mutated mtDNA molecules is greater than a reference value and results in the development of symptoms, such as the symptoms listed hereinbelow. This increased level of mutated mtDNA molecules may result in symptoms early in life (for example at birth, or in early childhood) or may lead to the development of symptoms later in life (for example when the subject reaches 10, 20, 30, 40 or more years of age). Methods for diagnosing mtDNA disorders are well known in the art. Such methods include measuring the level of mtDNA molecules in a muscle biopsy sample and/or enzyme activity assays (which may also be performed on a muscle biopsy sample). However, it will be appreciated that the method of the invention may provide a simpler, cheaper and/or less painful method for diagnosing mtDNA disorders.

Due to the fact that mtDNA disorders may be caused by mutations in many genes, these disorders are associated with a plethora of symptoms. The symptoms and their severity may differ from patient to patient (even when the same mtDNA mutation is present). By way of example, symptoms associated with mtDNA disorders may include poor growth, muscle weakness, muscle pain, low muscle tone, ataxia, exercise intolerance, partial or complete vison loss, partial or complete hearing loss, learning difficulties, delay in development, mental retardation, cardiomyopathy, epilepsy, liver dysfunction, kidney disfunction, and/or metabolic symptoms (such as lactic acidosis). Examples of mtDNA mutations and associated symptoms are described in Chinnery, P. F. (2014), however other symptoms will also be known to those skilled in the art.

Examples of mtDNA disorders include mitochondrial encephalopathy with lactic acidosis and stroke-like episodes (MELAS); chronic progressive external ophthalmoplegia (CPEO); myoclonic epilepsy with ragged-red fibers (MERRF); Leber's hereditary optic neuropathy (LHON), Leigh syndrome; Kearns-Sayre syndrome (KSS); neuropathy, ataxia, retinitis pigmentosa (NARP); Alpers-Huttenlocher syndrome; ataxia neuropathy syndromes (ANS) (such as MIRAS, SCAE, SANDO and myoclonic epilepsy myopathy sensory ataxia (MEMSA)); Pearson's syndrome; infantile myopathy and lactic acidosis (fatal and non-fatal forms); and progressive brain-stem disorder (MILS). Other mtDNA disorders will be known to those skilled in the art.

The term “diagnosis” or “diagnosing” as used herein refers to a method by means of which it can be determined whether the subject has or does not have a mtDNA disorder.

The term “prognosis” refers to the method by means of which a prediction of what will happen in the development or course of a mtDNA disorder can be made. In other words, it is understood as the expected evolution of a mtDNA disorder and refers to the prediction regarding the age of onset, state of development, evolution, or regression thereof, and/or the prognosis of the course of the mtDNA disorder in the future. It will be appreciated that a prediction might not always be accurate and/or informative. However, the term “prognosis” requires that the prediction be accurate and/or informative regarding the development or course of a diagnosed mtDNA disorder in at least some of the a statistically significant part of subjects, for example in at least 30%, at least 40%, at least 50%, at least 60%, at least 70%, at least 80%, at least 90%, at least 95%, or more of the subjects. It will be appreciated that the prognosis may be not solely based on the levels of mutated mtDNA molecules, and may be made in conjunction with, for example, other clinical observations, such as symptom severity, subject's age and family history. Suitably, the prognosis may provide an accurate and/or informative prognosis regarding the development or course of a diagnosed mtDNA disorder in a statistically significant part of the subjects. The amount that is statistically significant can be established by a person skilled in the art by using different statistical tools, for example, but not limited to, by determining confidence intervals, determining the significant p-value, Student's t-test or Fisher's discriminant function, non-parametric Mann-Whitney measurements, Spearman's correlation, logistic regression, linear regression, area under the ROC curve (AUC). Preferably, the confidence intervals are at least 90%, at least 95%, at least 97%, at least 98% or at least 99%. Preferably, the p-value is less than 0.1, than 0.05, than 0.01, than 0.005 or than 0.0001.

The term “determining the risk” as used herein refers to a method by means of which it can be determined whether the subject may develop a mtDNA disorder in the future. Such a subject may be typically asymptomatic but would have a level of mutated mtDNA molecules that may or may not result in the development of a mtDNA disorder. Whether the subject develops the disorder may depend, for example, upon the specific mutation type and/or the level of mutated mtDNA molecule.

The term “mutated mtDNA molecule” as used herein refers to a mtDNA molecule that has a mutation. In the context of the present specification, a “mutation” is any change in the mtDNA sequence or structure as compared to a wild-type mtDNA (non-mutated DNA), which results in the development of symptoms associated with a mtDNA disorder. Merely by way of example, the mutation may be a base alteration, a deletion, a strand break, an adduct formation, an insertion, a duplication, and/or altered methylation in mtDNA. It will be appreciated that mtDNA molecules may have polymorphisms (e.g., mutations which do not result in the development of symptoms associated with a mtDNA disorder). Such mtDNA molecules are referred to herein as “non-mutated mtDNA molecules” or “wild-type mtDNA molecules”. The sequence of the human wild-type mtDNA is well known in the art and may be found, for example, at GenBank accession number NC_012920.1 (31 Oct. 2014). Common polymorphisms in the wild-type mtDNA sequence are also well known. A list of such polymorphisms may be accessed through the MitoMap Database (mitomap.org, version r105; 20 Feb. 2020).

The mutation may be in a coding region of a mtDNA molecule and/or in a non-coding region of a mtDNA molecule.

Suitably, the non-coding region may be the heavy chain replication origin (O_{H}) and/or the light chain replication origin (O_{L}).

Suitably, the coding region may encode a tRNA, a protein and/or a rRNA gene.

The gene that encodes a tRNA may be selected from the group consisting of MT-TL1, MT-TA, MT-TC, MT-TD, MT-TE, MT-TF, MT-TG, MT-TH, MT-TI, MT-TK, MT-TL2, MT-TM, MT-TN, MT-TP, MT-TQ, MT-TR, MT-TS1, MT-TS2, MT-TT, MT-TV, MT-TW, and MT-TY.

The gene that encodes a protein may be selected from the group of MT-ATP6, MT-ATP8, MT-CO1, MT-CO2, MT-CO3, MT-CYB, MT-ND1, MT-ND2, MT-ND3, MT-ND4, MT-ND4L, MT-ND5, and MT-ND6. These genes encode proteins that are subunits of the mitochondrial respiratory chain complexes, specifically NADH: ubiquinone oxidoreductase (complex I), ubiquinol:cytochrome c oxidoreductase (complex III), cytochrome c oxidase (complex IV), or ATP synthase (complex V). Accordingly, the mutation may be in a gene that encodes a subunit of a mitochondrial respiratory chain complex selected from the group consisting of complex I, complex III, complex IV and complex V.

The gene that encodes a rRNA may be selected from the group of MT-RNR1 and MT-RNR2.

It will be appreciated that some mutation, such as deletions, can span across larger parts of a mtDNA molecule. Accordingly, such mutations may be in a coding and non-coding region, and/or can be in more than one gene. More suitably, the mtDNA disorder may be caused by a mutation in the gene MT-TL1. By way of example, the mutation in MT-TL1 may be m.3243A>G.

Many mutations in a mtDNA molecule may cause a mtDNA disorder. The MitoMap Database (mitomap.org, version r105; 20 Feb. 2020) provides a non-limiting list of disease-causing mutations. The skilled person will appreciate that the methods described herein may be utilised to determine the level of mutated mtDNA molecules having any of the mutations listed in the MitoMap Database. Merely by way of example, a mtDNA mutation may be selected from the group consisting of m.3243A>G, m.3243A>T, m.3244G>A, m.3251A>G, m.3255G>A, m.3256C>T, m.3273T>C, m.3283G>A, m.3302A>G, m.3460G>A, m.4267A>G, m.4272T>C, m.4284G>A, m.4298G>A, m.4302A>G, m.4308G>A, m.4309G>A, m.4315delA, m.5532G>A, m.5628T>C, m.5636T>C, m.5814T>C, m.7458G>A, m.8342G>A, m.8344G>A, m.8355T>C, m.8363G>A, m.8993T>G/C, m.11323T>C, m.11778G>A, m.12276G.A, m.12294G>C, m.12315G>A, m.12316G.A, m.14484T>C, m.14710G>A, m.14723T>C, m.582T>C, m.611G>A, and m.642T>C.

As mentioned, the methods described herein may be in vitro methods that are performed using a stool sample that has already been obtained from the subject (i.e. the sample is provided for the method, and the steps taken to obtain the sample from the subject are not included as part of the method). The methods may therefore include the step of providing a stool sample from a subject. As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample, or being provided the sample by the subject. Indirectly obtaining a sample refers to receiving the sample from another party or source (e.g., a third party laboratory that directly acquired the sample).

The term “stool sample” used herein should be understood to mean faecal matter obtained from the subject. Such a sample may be collected non-invasively after the subject defecates. In a subject that is a baby, the sample may be collected from the nappy. The terms “stool sample” and “faecal sample” may be used herein interchangeably.

The methods described herein include the step of assaying a stool sample from the subject to measure the level of mutated mtDNA molecules.

Suitably, assaying may include extraction of mtDNA from the sample. The step of extraction may be to purify the mtDNA from contaminants (such as bacterial DNA). Merely by way of example, mtDNA may be extracted using the FastDNA® SPIN Kit for Feces (MpBio). Suitably mtDNA may be extracted from a stool sample having a weight of from about 50 mg to about 1000 mg, from about 100 mg to about 500 mg, from about 200 mg to about 300 mg. Suitably the mtDNA may be extracted from a stool sample weighing about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg or about 1000 mg, or more. An exemplary method of extracting mtDNA is provided in the Examples section of the present specification.

Suitably, assaying may include sequencing mtDNA molecules present in the sample, or fragments thereof. The fragments may correspond to parts of the mtDNA molecules in which a mutation is suspected (for example based on the subject's symptoms and/or family genetic history). The term fragment as used herein refers to a nucleic acid that is shorter than the whole length of mtDNA. Suitably, the fragment may be about 20, about 30, about 40, about 50, about 60, about 70, about 80, about 90, about 100, about 150, about 200, about 300, about 400 or more base pairs in length. In the present specification, the term “mtDNA”, “mtDNA molecule”, and “mtDNA molecules” are used interchangeably, and refer to the DNA found insider the mitochondria.

Sequencing may be performed by any suitable sequencing method. Merely by way of example sequencing may be by pyrosequencing, whole genome sequencing, and/or Sanger sequencing. Exemplary methods of performing pyrosequencing next and whole genome sequencing are provided in the Examples section of the present specification.

Assaying may include amplification of the mtDNA molecules or fragments thereof. Suitably, amplification may be prior to sequencing. In such an example, sequencing may be of the amplified mtDNA molecules or fragments thereof.

Merely by way of example, amplification may be by polymerase chain reaction (PCR), loop mediated isothermal amplification, nucleic acid sequence-based amplification, strand displacement amplification, rolling circle amplification and ligase chain reaction. An exemplary method of performing PCR is provided in the Examples section of the present specification. It will be appreciated that sequencing and/or amplification may be after extraction of mtDNA from the sample.

Assaying may further comprise data analysis to determine the level of mutated mtDNA molecules. Suitably, the level of mutated mtDNA molecules may be determined as a proportion of mutated mtDNA molecules to non-mutated mtDNA molecules, a percentage of mutated mtDNA molecules in the sample, and/or a number of mutated mtDNA molecules in a sample.

In the context of the methods of the present invention, typically the greater the level of mutated mtDNA, the greater the risk that the subject will develop a mtDNA disorder, that the disorder will be associated with more severe symptoms, and/or that the age of onset of the mtDNA disorder will be lower. Levels of mutated mtDNA associated with severity of symptoms, age of onset and/or risk of developing a mtDNA disorder will be well known to those skilled in the art and widely discussed in literature (for example in Grady et al. EMBO Mol Med. 2018 June; 10(6):e8262; Pickett et al. Ann Clin Transl Neurol. 2018 Feb. 7; 5(3):333-345; Gorman et al. Nat Rev Dis Primers. 2016 Oct. 20; 2:16080) in the context of heteroplasmy in muscle tissue. It will be appreciated that these equally apply to the methods described herein due to substantially the same heteroplasmy levels in muscle and stool samples as found by the inventors of the present invention.

Accordingly, a method for providing a diagnosis or prognosis for a mtDNA disorder or for determining the risk of a mtDNA disorder developing in a subject as described herein may comprise
a) assaying a stool sample from the subject to measure the level of mutated mtDNA molecules; optionally wherein assaying comprises sequencing mtDNA molecules or fragments thereof, further optionally wherein the mtDNA molecules or fragments thereof are amplified prior to sequencing, and/or wherein the mtDNA molecules are extracted from the stool sample prior to sequencing or amplification; and
b) comparing the level to a reference value, wherein an increase in the level as compared to the reference value is indicative of the mtDNA disorder or of an increased risk of the mtDNA disorder developing in the subject.

The method may further include the step of administering the subject that has been diagnosed with a mtDNA disorder or determined to be at risk of developing a mtDNA disorder with a therapeutically effective amount of a treatment for a mtDNA disorder. Many suitable treatments will be known to those skilled in the art. A suitable treatment may depend upon the mutated gene, mutation, subject's symptoms, and/or family history. Exemplary treatments are mentioned elsewhere in the present specification. The term “therapeutically effective amount” as used herein refers to an amount of treatment that has a therapeutic effect on the subject as described elsewhere in the present specification.

The phrase “level of mutated mtDNA molecules” may be used interchangeably with phrases such as “levels of heteroplasmy”, “heteroplasmy levels” or “mutation load” in the present specification and refer to the amount of mutated to non-mutated mtDNA molecules in the sample. The mutated mtDNA molecules may have the same or different disease-causing mutations (i.e. mutations that result in a mtDNA disorder or put the subject at risk of developing a mtDNA disorder). It will be appreciated that non-mutated mtDNA molecules may also have mutations, however these mutations are not disease causing (i.e. do not result in a mtDNA disorder, nor do they put the subject at risk of developing a mtDNA disorder).

As used herein, the term “reference value” refers to a level of mutated mtDNA molecules that does not result in a mtDNA disorder and/or does not put the subject at risk of developing a mtDNA disorder. The reference value may be a specific value (for example about 10%, about 20%, about 30%, about 40%, about 50%, about 60%, about 70%, about 80%, about 90%, etc) or a range (for example from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, etc). The reference value may be the level of mutated mtDNA molecules in a sample from a healthy subject. It will be appreciated that in the context of the present specification, “a healthy subject” refers to someone who does not have a mtDNA disorder or is not at risk of developing a mtDNA disorder despite having some level of mutated mtDNA molecules. The reference value may be based on heteroplasmy levels in any suitable sample from a healthy subject, such as a muscle sample, a stool sample and/or a urine sample. Preferably, the reference value may be based on heteroplasmy levels measured in muscle biopsy and found to not result in a mtDNA disorder and/or not put the subject at risk of developing a mtDNA disorder.

The reference value may be a predetermined value. Accordingly, it will be appreciated that the method for providing diagnosis and/or prognosis for a mtDNA disorder or for determining the risk of a mtDNA disorder developing in a subject does not require the step of measuring the levels of mutated mtDNA molecules in a healthy subject. Such a step, however, may be included in said methods. Measuring the levels of mutated mtDNA molecules in a healthy subject may be useful, for example, when the subject has a novel mutation in mtDNA a predetermined reference value does not exist.

It will be appreciated that the reference value may differ depending on the mutation in question. The skilled person will recognize that certain mutations have a higher tolerability, whilst others have a lower tolerability. Therefore, merely by way of example, for some mtDNA mutations the reference value may be 10% or less (for example 0%), whilst for others the reference value may be 50% or more. For example, in the context of the mutation m.3243A>G, the reference value may be 40%, or more.

Those skilled in the art will appreciate that mtDNA disorders are a group of complex diseases with large subject to subject variation. As such, the level of mutated mtDNA molecules in a subject might not always directly correlate with the severity of the subject's symptoms, the subject's risk of developing a mtDNA disorder and/or age of disease onset. Accordingly, the levels of mutated mtDNA molecules may be used in combination with clinical observations, other laboratory results, and/or family history in order to determine whether the subject has a mtDNA disorder or is at risk of developing such a disorder.

The reference value may be the level of heteroplasmy in a single healthy subject or a group of such subjects. The group may comprise, at least 5, 10, 50, 100, 1000 or more healthy subjects. Suitably, when the reference value is the heteroplasmy level in a group of people, the reference value may be the median or mean heteroplasmy level in said group.

In the methods described herein the subject may be identified as having a mtDNA disorder or as being at risk of developing a mtDNA disorder if the comparison of the subject's level of heteroplasmy (the level of heteroplasmy being determined in a stool sample from the patient) to a reference value indicates that the subject has an increased level of heteroplasmy than the reference value.

The term “increased” or “increase” as used herein generally means a difference between the subject's level of mutated mtDNA to a reference value, that is at least about 10% greater than the reference value, for example at least about 20%, at least about 30%, at least about 40%, at least about 50%, at least about 60%, at least about 70%, at least about 80%, at least about 90% greater than the reference value. It will be appreciated that where the reference value is a reference range, the increase may be as compared to the lower end or upper end of the reference range. When the increase is as compared to a lower end of the reference range, the skilled person may determine if the subject has or is at risk of developing a mtDNA disorder by combining the heteroplasmy results with clinical observations, other laboratory results, and/or family history. For example, a subject may be diagnosed to have a mtDNA disorder if heteroplasmy levels are higher as compared to the lower end of the reference range but lower than the higher end of the reference range, when the subject shows symptoms associated with a mtDNA disorder and/or other laboratory investigations (for example mitochondrial respiratory enzyme assays) are indicative of a mtDNA disorder.

In a further aspect, the present invention relates to a method of providing a diagnosis and/or prognosis for a mtDNA disorder or for determining the risk of a mtDNA disorder developing in a subject, and treating or preventing the mtDNA disorder, the method comprising the steps of:
a) assaying a stool sample from the subject to measure the level of mutated mtDNA molecules;
b) comparing the level to a reference value, wherein an increase in the level as compared to the reference value is indicative of the mtDNA disorder or of an increased risk of the mtDNA disorder developing in a subject; and
c) administering the subject that has been identified as having or being at risk of having mtDNA disorder a treatment for mtDNA disorder, thereby treating the subject.

Suitably, the method of providing a diagnosis and/or prognosis for a mtDNA disorder or for determining the risk of a mtDNA disorder developing in a subject, and treating or preventing the mtDNA disorder, may comprise the steps of:
a) assaying a stool sample from the subject to measure the level of mutated mtDNA molecules; optionally wherein assaying comprises sequencing mtDNA molecules or fragments thereof, further optionally wherein the mtDNA molecules or fragments thereof are amplified prior to sequencing, and/or wherein the mtDNA molecules are extracted from the stool sample prior to sequencing or amplification;
b) comparing the level to a reference value, wherein an increase in the level as compared to the reference value is indicative of the mtDNA disorder or of an increased risk of the mtDNA disorder developing in the subject; and
c) administering the subject that has been identified as having or being at risk of having mtDNA disorder a treatment for mtDNA disorder, thereby treating the subject.

It will be appreciated that in an embodiment, wherein the mtDNA molecules are extracted from the stool sample prior to sequencing of amplification, the method may comprise the steps of:
a) extracting mtDNA molecules from the stool sample;
b) optionally, amplifying the extracted mtDNA molecules or fragments thereof;
c) assaying the mtDNA molecules to measure the level of mutated mtDNA molecules, optionally wherein assaying comprises sequencing mtDNA molecules or fragments thereof;
d) comparing the level to a reference value, wherein an increase in the level as compared to the reference value is indicative of the mtDNA disorder or of an increased risk of the mtDNA disorder developing in the subject; and
e) administering the subject that has been identified as having or being at risk of having mtDNA disorder a treatment for mtDNA disorder, thereby treating the subject.

In another aspect, the present invention provides a method of treating a mtDNA disorder in a subject, the method comprising:
a) requesting a test providing the results of an analysis to determine the level of mutated mtDNA molecules in a stool sample from the subject and comparing the level to a reference value, wherein an increase in the level as compared to the reference value is indicative of the mtDNA disorder or of an increased risk of the mtDNA disorder developing in a subject; and
b) administering the subject that has been identified as having or being at risk of having mtDNA disorder a treatment for mtDNA disorder, thereby treating the subject.

Suitably, the test may be performed by a party other than the party requesting the test.

Suitably, the test may be performed by assaying a stool sample as described elsewhere in the present specification.

The term “administering”, and the like, as used herein, refer to delivery of a treatment for a mtDNA disorder. Suitably, the treatment may be administered in a therapeutically effective amount. Administration may be by any suitable route, for example intravenous, intramuscular, intradermal, intraperitoneal, subcutaneous, oral, topical, intrathecal, inhalational, intranasal, transdermal, rectal, and the like.

Methods for Evaluating a Therapeutic Effect of a Treatment for mtDNA Disorder

In one aspect, the invention provides a method for evaluating a therapeutic effect of a treatment for a mtDNA disorder, the method comprising the steps of:
a) assaying a stool sample from the subject before treatment to measure the level of mutated mtDNA molecules;
b) assaying a stool sample from the subject after treatment to measure the level of mutated mtDNA molecules; and
c) comparing the level obtained in step a) and step b), wherein a decrease in the level obtained in step b) as compared to the level in step a) is indicative of the treatment having therapeutic effect.

Suitably, the method may be an in vitro method for evaluating a therapeutic effect of a treatment for mtDNA disorder.

Assaying in steps a) and b) may be performed as discussed elsewhere in the present specification.

The phrase “a stool sample from the subject before treatment” refers to a stool sample that has been obtained from the subject at a time point before the treatment for the mtDNA disorder has begun, at the same time as commencing the treatment (for example on the same day), or at a time point after the treatment has begun but not finished. Thus, the method can be used to determine the therapeutic effect of a treatment for a mtDNA disorder from the outset (i.e. from the start of the treatment period) or from a time point after the treatment has started (i.e. determining or monitoring the therapeutic effect of a treatment for a mtDNA during the treatment itself).

The phrase “a stool sample from the subject after treatment” refers to a stool sample that has been obtained from the subject at a time point after treatment for the mtDNA disorder has begun, optionally at a time point when treatment has finished (i.e. at the end of the treatment period). The term “treatment period” refers to a time interval over which treatment occurs (e.g. 1 month, 3 months, 6 months, 1 year, 2 years, etc). It will be appreciated that a stool sample from the subject after treatment will have been obtained from the subject at a later time point than “a stool sample from the subject before treatment”.

The method for evaluating a therapeutic effect of a treatment for mtDNA disorder as described herein may be a useful screening tool for identifying potential treatments for mtDNA disorders, for example in the context of clinical trials.

The term “treatment” as used herein refers to a compound and/or regimen that has or is believed to have therapeutic effect (for example on the basis of in vitro or in vivo studies) in a subject with a mtDNA disorder. The compound and/or regimen may be new or may be known but needs further testing (for example to determine if it has a therapeutic effect on a particular patient or group of patients). The compound may be, for example, a small molecule, protein or nucleic acid. The term “regimen” as used herein refers to the dosage, frequency of administration, and/or length of time the subject receives the compound.

The term “decrease” or “decreased” as used herein, generally means a reduction in the subject's level of mutated mtDNA molecules. The reduction may be by at least about 5%, at least about 10%, at least about 20%, at least about 30%, at least about 40%, at least about 50%, at least about 60%, at least about 70%, at least about 80%, at least about 90% or more.

The term “therapeutic effect” as used herein refers to any beneficial effect that the treatment may have on a subject with a mtDNA disorder. Such a beneficial effect may be, for example, reduction or elimination of symptoms of a mtDNA disorder, delay of symptom onset, and/or decrease in the level of mutated mtDNA molecules. Exemplary symptoms associated with mtDNA disorders are discussed elsewhere in the present specification.

The method for evaluating a therapeutic effect of a treatment for mtDNA disorder may also be useful for determining the subject's compliance with a prescribed treatment for a mtDNA disorder. This gives rise to a further aspect of the invention, directed to a method for determining a subject's compliance with a prescribed treatment for a mtDNA disorder, the method comprising:
a) assaying a stool sample from the subject before treatment to measure the level of mutated mtDNA molecules;
b) assaying a stool sample from the subject after treatment to measure the level of mutated mtDNA molecules; and
c) comparing the level obtained in step a) and step b), wherein a decrease in the level obtained in step b) as compared to the level in step a) indicates that the subject has adhered to a prescribed treatment.

Unless required otherwise by the context, embodiments and examples described in relation to a method for evaluating a therapeutic effect of a treatment for a mtDNA disorder also apply to a method for determining a subject's compliance with a prescribed treatment for a mtDNA disorder. This is because a “prescribed treatment” is a recommended treatment and therefore typically has a therapeutic effect (and thus, observation of the therapeutic effect on the symptoms and/or heteroplasmy levels is an indication of subject compliance with the prescribed treatment regimen). The prescribed treatment may be, for example, coenzyme Q10, B complex vitamins (such as thiamine (B1) and riboflavin (B2)), alpha lipoic acid, L-carnitine, creatine, L-Arginine, endurance exercise, and/or resistance training (also known as strength training).

Methods for Monitoring the Progression of a mtDNA Disorder

In one aspect, the invention provides a method for monitoring the progression of a mtDNA disorder in a subject, the method comprising the steps of:
a) assaying a stool sample from the subject to measure the level of mutated mtDNA molecules;
b) repeating step a) for the same subject after a time interval; and
c) comparing the level measured in step a) and step b), wherein a change in the levels measured in a) and b) indicates a change in the progression of the mtDNA disorder in the subject.

Suitably, the subject monitored for the progression of a mtDNA disorder may be asymptomatic.

The change may be a decrease or an increase in the levels measured in step b) as compared to the levels measured in step a). An increase may be indicative of the fact that the mtDNA disorder is progressing (which may coincide with the subject having or being likely to develop more severe symptoms and/or show symptoms at a younger age). By the same token, a decrease in the levels may be indicative of the fact that the disorder is regressing. Regression may be spontaneous or as a result of a treatment having a therapeutic effect, as described hereinabove.

Suitable time intervals for monitoring progression of a mtDNA disorder can easily be identified by a person of skill in the art and will depend on the specific form of mutation and/or heteroplasmy levels. As a non-limiting example, the time interval may be six months, a year, or whenever clinically needed, i.e. in case of a significant change in the symptoms associated with a mtDNA disorder. For the sake of clarity, it will be appreciated that the stool sample in step b) will have been produced by the subject at a later time point than the sample used in step a).

The present invention also provides the use of a stool sample in a method as described herein. Suitably, the use may be in a clinical trial for a mtDNA disorder treatment.

It will be appreciated that the details of assaying, subjects, mtDNA disorders, levels of mutated mtDNA molecules, etc., discussed in the context of the method for providing a diagnosis or prognosis of a mitochondrial DNA (mtDNA) disorder or for determining the risk of a mtDNA disorder developing in a subject also apply to the method for evaluating a therapeutic effect of a treatment for mtDNA disorder, a method for determining a subject's compliance with a prescribed treatment for a mtDNA disorder, a method for monitoring the progression of a mtDNA and uses of the invention.

### EXAMPLES

### 1. Introduction

Mitochondrial disorders are a group of genetic disorders that are highly heterogeneous, with substantial variability in disease burden and progression, although almost every organ in the body is affected¹. A deeper understanding of the genetics and molecular basis of mitochondrial disease, alongside advances in sequencing technologies has dramatically transformed clinical management of mitochondrial disorders². Despite these advances, reasons for this heterogeneity is poorly understood, consequently, clinical management is made more difficult due to the tenuous link between a patient's clinical phenotype and genotype, and clinical outcomes and long term prognosis is difficult to predict³.

### 2. Materials and Methods

### 2.1 Study Population

Two stools were collected 12 weeks apart from seventeen adult m3243A>G carriers (14 females) recruited from the NHS highly Specialised Service for Rare Mitochondrial Disorders or Adults and Children in Newcastle Upon Tyne.

### 2.2 DNA Extraction

The Mitochondrial DNA (mtDNA) was extracted from 250 mg of stool for both samples using the FastDNA® SPIN Kit for Feces (MpBio) per the manufacturers' protocol. Briefly, 250 mg of stool, 825 μl sodium phosphate buffer and 275 μl of PLS solution was added to a lysing matrix tube and mixed. Samples were then centrifuged, and the supernatant was removed. 978 and 122 μl of sodium phosphate buffer and MT buffer were added to the pellet and vortexed. Samples were then placed in a FastPrep® for 40 seconds at 6 m/s, followed by centrifugation at 14,000×g for 15 minutes. The supernatant was then transferred to a clean 2 ml Eppendorf® containing 250 μl of PPS solution. This was then gently mixed and stored at 4° C. for 10 minutes and then centrifuged at 14,000×g for 2 minutes. The supernatant was then added to a 15 ml conical containing 1 ml of binding matrix solution and then placed on a rocker for 5 minutes. The samples were then centrifuged at 14,000×g for 2 minutes, the supernatant was removed and 1 ml of washing butter was then mixed with the pellet. Following mixing, the supernatant was then transferred to a SPIN filter tube and then centrifuged at 14,000×g for 1 minute. The catch tube was then emptied, and the pellet was re-suspended using 500 μl Wash Buffer 2, followed by centrifugation at 14,000×g for 2 minutes. The SPIN filter was then transferred to a new Eppendorf® and 30 μl of TES was added. The sample was then centrifuged at 14,000×g for 2 minutes to elute the pure DNA ready for polymerase chain reaction.

### 2.3 Amplification of DNA

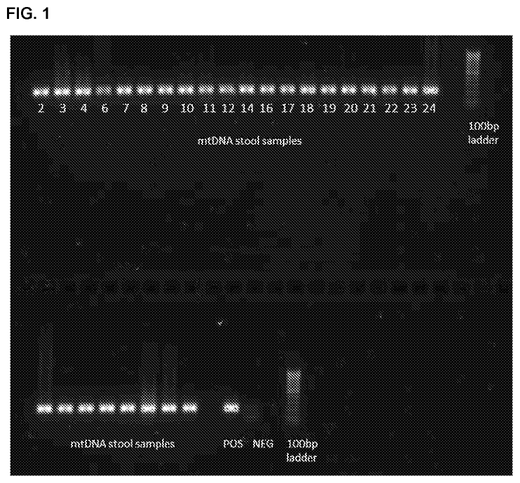

A single stage PCR reaction was used to amplify a region of the mtDNA comprising the m.3243 A>G mutation from mtDNA extracted from stool samples. mtDNA in 25 μl volumes using a mastermix comprising of; nuclease free water (9.8 μl/sample), 5× buffer (5 μl/sample, Promega, M890) magnesium chloride at 25 mM (2 μl, Promega, A351H), deoxyribonucleotide triphosphate X10 (2.5 μl/sample), GoTaq® HotStart Polymerase at 5 μl/μl (0.2 μl/sample, Promega, M7408), forward and reverse primers at 10 μmol/L (1.25 μl/sample) were used to amplify the 210 base pair fragment of mtDNA using the following primers 5′/5Biosg/TAA GGC CTA CTT CAC AAA GCG 3 (SEQ ID NO: 1) and 5′ GCG ATT AGA ATG GGT ACA ATG AG 3′ (SEQ ID NO: 2) respectively) totalling 23 μl. 2 μl of mtDNA was added to the respective tubes, including a positive and negative control. The reaction conditions were 95° C. for 2 minutes, followed by 36 cycles of 95° C. for 30 seconds, 63° C. for 30 seconds, 72° C. for 30 seconds, followed by 72° C. for 10 minutes and 4° C. for 10 minutes. PCR products were then run on electrophoresis using a 1% agarose gel for 40 minutes and band sized against a 100 bp ladder (Promega) (FIG. 1).

2.4 Pyrosequencing of mtDNA

To measure levels of m.3243A>G heteroplasmy in mtDNA pyrosequencing was used. Briefly, PyroMark Q24 software was used to design the plate with sample IDs, negative and positive samples. Binding buffer master mix was prepared using nuclease free water (28 μl/sample), binding buffer (40 μl/sample, 1056479, Qiagen) and sepharose beads (2 μl/sample, 17-5113-01, GE healthcare). 70 μl of the binding buffer master mix was added to the appropriate wells, followed by 10 μl of PCR product. The plate was sealed and vortexed for 10 minutes at room temperature. Annealing buffer master mix was prepared using annealing buffer (24.25 μl/sample) and pyrosequencing primer (0.75 μl/sample at 10 μmol/L) according to GenBank Accession number NC_012920.1: 5 ′biotinylated forward: m.3143-3163; reverse: m.3331-3353; and reverse pyrosequencing primer: m.3244-3258 (IDT, Coralville). Samples and sepharose beads were taken up using the hedgehog, followed by 5 seconds in ethanol, denaturation buffer (0.2M sodium hydroxide) and washing buffer. Sephrose beads were then placed into the second tray containing annealing buffer and mixed for 10 seconds. This tray was then placed on a heating block at 80° C. for 2 minutes, and then placed into the Pyromark Q24 platform. Pyrogold nucleotides, substrate and enzymes (970802, Qiagen) were then added to the Pyromark Q24 cartridge and the run was started. Allele quantification application of Pyromarks Q24 software was used to calculate heteroplasmy levels (test sensitivity >3% mutant mtDNA).

### 2.5 Whole Genome Sequencing

### 2.5.1 PCR Amplification

To exclude the possibility of nuclear pseudogene amplification, DNA (20-50 ng) was amplified with two overlapping primer pair sets, generating 2-9 kb amplicons as previously described in Greaves, Nooteboom⁴, using GoTaq Long PCR Master Mix (Promega, Southampton, UK) according to the manufacturer's protocol. PCR products were purified with AMPure XP Reagent (Beckman Coulter, High Wycombe, UK) and subsequently quantified on an Agilent Bioanalyzer 2100 using a DNA 12,000 kit (Agilent Technologies, Stockport, UK).

2.5.2 mtDNA Sequencing

Overlapping mtDNA PCR amplicons were pooled in equimolar concentrations to 100 ng and then fragmented, barcoded, size-selected and amplified using the IonXpress Plus Fragment Library kit, Ion Xpress Barcode Adapters 1-16 kit and E-Gel SizeSelect 2% agarose gels (ThermoFisher Scientific, Paisley, UK), as per the manufacturer's instructions. Barcoded libraries were assessed on the 2100 Bioanalyzer using an Agilent High Sensitivity DNA kit, then pooled in equimolar concentrations. Clonal amplification of pooled libraries (20 pM), enrichment and loading onto Ion 316 v2 BC sequencing chips was performed on an Ion Chef instrument using the Ion PGM HI-Q View Chef kit, according to the manufacturer's recommendations (ThermoFisher Scientific). Next generation sequencing was performed on the Ion Torrent Personal Genome Machine (Life Technologies), running Torrent Suite version 5.10.2, using the Ion PGM HI-Q View Seq kit (ThermoFisher Scientific). Data analysis was carried out with the Torrent Suite variantCaller v5.10.1.20 (mtDNA custom settings). All detected mtDNA variants were manually reviewed with the Integrative Genomics Viewer (v2.4.19, Broad Institute, Cambridge, Mass., US).

### 2.6 Statistics

Statistical analysis was performed in R⁵. Data for levels of heteroplasmy in blood are presented as corrected and uncorrected as previously described⁶. Briefly, blood heteroplasmy was adjusted to account for a known decline with age.

### 3. Results

Inter-reliability revealed that on average, mtDNA levels of heteroplasmy from stool samples was 64±3% and 63±3% for stool sample 1 and 2 respectively. This difference −0.94, bias-corrected and accelerated (BCa) 95% confidence interval (CI) [−4.57, 2.69], was not significant t (17)=−0.50, p=0.60 (FIG. 1). A second independent researcher repeated the same experiment and reported on average, mtDNA levels of heteroplasmy from stool samples was 62±4% and 63±3% for stool sample 1 and 2 respectively. This difference −0.77, bias-corrected and accelerated (BCa) 95% confidence interval (CI) [−3.53, 1.99], was not significant t (17)=−0.60, p=0.56

### 4. Analysis

Intra reliability was assessed by two independent researchers, comparing stool sample 1 vs stool sample 1 and stool sample 2 vs stool sample 2, with both being blinded to results. On average, mtDNA levels of heteroplasmy for stool sample 1 were 64±3% and 62±4%. This difference 0.12, bias-corrected and accelerated (BCa) 95% confidence interval (CI) [−1.54, 1.78], was not significant t (17)=−0.15, p=0.88. On average, mtDNA levels of heteroplasmy for stool sample 2 were 63±3% and 64±3%. This difference −0.82, bias-corrected and accelerated (BCa) 95% confidence interval (CI) [−1.70, 0.05], was not significant t (17)=−1.99, p=0.06.

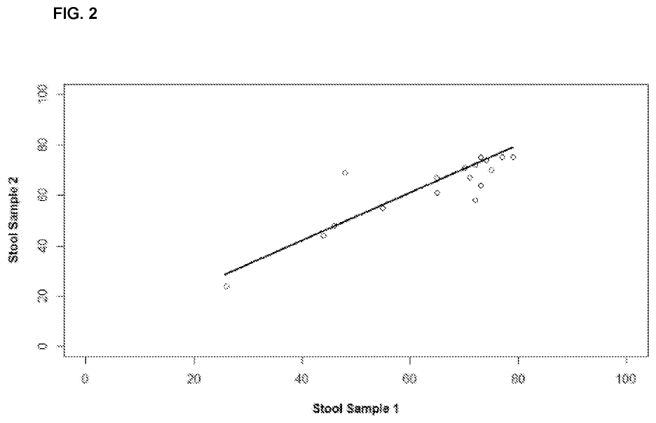

A mixed effect model was used to compare different methods of assessing levels of heteroplasmy, whilst accounting for patient ID as a random effect. Muscle was used as a reference gold standard to assess heteroplasmy, and all estimates are relative to this. Unadjusted and adjusted blood heteroplasmy were significantly different from muscle heteroplasmy (p=<0.01), however, there were no other significant differences between muscle and other measures of heteroplasmy (p=>0.05) (FIG. 2). Repeated measures of mtDNA heteroplasmy from stool samples by an independent researcher were also not significantly different from any other measures of heteroplasmy (p=>0.05). However, unadjusted and adjusted blood heteroplasmy were significantly different from muscle and other measures of heteroplasmy (p=<0.01).

### REFERENCES

1. Gorman G S, Chinnery P F, DiMauro S, et al. Mitochondrial diseases. Nat Rev Dis Primers. 2016; 2:16080.
2. Alston C L, Rocha M C, Lax N Z, Turnbull D M, Taylor R W. The genetics and pathology of mitochondrial disease. J Pathol. 2017; 241(2):236-50.
3. Chinnery P F, Howell N, Lightowlers R N, Turnbull D M. Molecular pathology of MELAS and MERRF. The relationship between mutation load and clinical phenotypes. Brain. 1997; 120 (Pt 10):1713-21.
4. Greaves L C, Nooteboom M, Elson J L, et al. Clonal expansion of early to mid-life mitochondrial DNA point mutations drives mitochondrial dysfunction during human ageing. PLoS Genet. 2014; 10(9):e1004620.
5. R Development Core Team. R: A language and environment for statistical computing. Vienna, Austria: R Foundation for Statistical Computing; 2010.
6. Grady J P, Pickett S J, Ng Y S, et al. mtDNA heteroplasmy level and copy number indicate disease burden in m.3243A>G mitochondrial disease. EMBO Mol Med. 2018; 10(6).
7. Chinnery P F. Mitochondrial Disorders Overview. 2000 Jun. 8 [Updated 2014 Aug. 14]. In: Adam M P, Ardinger H H, Pagon R A, et al., editors. GeneReviews® [Internet]. Seattle (Wash.): University of Washington, Seattle; 1993-2020.

1. A method for providing a diagnosis and/or prognosis for a mitochondrial DNA (mtDNA) disorder or determining the risk of a mtDNA disorder developing in a subject, the method comprising the steps of:
a) assaying a stool sample from the subject to measure the level of mutated mtDNA molecules; and
b) comparing the level to a reference value, wherein an increase in the level as compared to the reference value is indicative of the mtDNA disorder or of an increased risk of the mtDNA disorder developing in a subject.

2. A method of providing a diagnosis and/or prognosis for a mtDNA disorder or for determining the risk of a mtDNA disorder developing in a subject, and treating or preventing the mtDNA disorder, the method comprising the steps of:
a) assaying a stool sample from the subject to measure the level of mutated mtDNA molecules;
b) comparing the level to a reference value, wherein an increase in the level as compared to the reference value is indicative of the mtDNA disorder or of an increased risk of the mtDNA disorder developing in a subject; and
c) administering the subject that has been identified as having or being at risk of having mtDNA disorder a treatment for mtDNA disorder, thereby treating the subject.

3. A method for treating a mtDNA disorder in a subject, the method comprising:
a) requesting a test providing the results of an analysis to determine the level of mutated mtDNA molecules in a stool sample from the subject and comparing the level to a reference value, wherein an increase in the level as compared to the reference value is indicative of the mtDNA disorder or of an increased risk of the mtDNA disorder developing in a subject; and
b) administering the subject that has been identified as having or being at risk of having mtDNA disorder a treatment for mtDNA disorder, thereby treating the subject.

4. The method of claim 1, wherein the reference value is the level of mutated mtDNA molecules in a sample from a healthy subject.

5. The method of claim 4, wherein the healthy subject does not have mtDNA disease or is not at risk of developing mtDNA disease.

6. The method of claim 1, wherein the sample from the healthy subject is selected from the group consisting of a stool sample, a muscle biopsy sample, a blood sample and a urine sample.

7. A method for evaluating therapeutic effect of a treatment for a mtDNA disorder, the method comprising the steps of:
a) assaying a stool sample from a subject before treatment to measure the level of mutated mtDNA molecules;
b) assaying a stool sample from the subject after treatment to measure the level of mutated mtDNA molecules; and
c) comparing the level obtained in step a) and step b), wherein a decrease in the level obtained in step b) as compared to the level in step a) is indicative of the treatment having therapeutic effect.

8. A method for determining a subject's compliance with a prescribed treatment for a mtDNA disorder, the method comprising:
a) assaying a stool sample from the subject before treatment to measure the level of mutated mtDNA molecules;
b) assaying a stool sample from the subject after treatment to measure the level of mutated mtDNA molecules; and
c) comparing the level obtained in step a) and step b), wherein a decrease in the level obtained in step b) as compared to the level in step a) indicates that the subject is/and or has complied with the prescribed treatment.

9. A method for monitoring the progression of a mtDNA disorder in a subject, the method comprising the steps of:
a) assaying a stool sample from the subject to measure the level of mutated mtDNA molecules;
b) repeating step a) for the same subject after a time interval; and
c) comparing the level measured in step a) and step b), wherein a change in the levels measured in a) and b) indicates a change in the progression of the mtDNA disorder in the subject.

10. The method of claim 1, wherein assaying comprises extracting the mtDNA molecules from the sample.

11. The method of claim 1, wherein assaying comprises amplifying mtDNA molecules or fragments thereof.

12. The method of claim 11, wherein amplifying is by a method selected from the group consisting of polymerase chain reaction, loop mediated isothermal amplification, nucleic acid sequence based amplification, strand displacement amplification, rolling circle amplification and ligase chain reaction.

13. The method of claim 1, wherein the method comprises sequencing mtDNA molecules or fragments thereof.

14. The method of claim 1, wherein sequencing is by pyrosequencing, whole genome sequencing, and/or Sanger sequencing.

15. The method of claim 1, wherein the level of mutated mtDNA molecules is determined by the proportion of mutated mtDNA molecules to non-mutated mtDNA molecules in the sample, the percentage of mutated mtDNA molecules in the sample, or the number of mutated mtDNA molecules in the sample.

16. The method of claim 1, wherein the subject is a mammal.

17. The method of claim 16, wherein the subject is a child.

18. The method of claim 1, wherein the mtDNA disorder is selected from the group consisting of mitochondrial encephalopathy with lactic acidosis and stroke-like episodes (MELAS); chronic progressive external ophthalmoplegia (CPEO); myoclonic epilepsy with ragged-red fibers (MERRF); Leber's hereditary optic neuropathy (LHON), Leigh syndrome; Kearns-Sayre syndrome (KSS); neuropathy, ataxia, retinitis pigmentosa (NARP); Alpers-Huttenlocher syndrome; ataxia neuropathy syndromes (ANS); Pearson's syndrome; infantile myopathy and lactic acidosis (fatal and non-fatal forms); and progressive brain-stem disorder (MILS).

19. The method of claim 1, wherein the mutated mtDNA comprises a mutation in a gene and/or in a non-coding region of the mtDNA.

20. The method of claim 18, wherein the gene encodes a tRNA, a protein and/or a rRNA.

21. The method of claim 19, wherein the gene encoding a tRNA is MT-TL1.

22. The method of claim 20, wherein the mutation is m.3243A>G.

23. The method of claim 2, wherein the treatment is selected from the group consisting of coenzyme Q10, B complex vitamins, alpha lipoic acid, L-carnitine, creatine, L-Arginine, endurance exercise, and resistance training.

24. Use of a stool sample in a method as defined in claim 1.

25. The method of claim 16, wherein the subject is a human, a mouse, a rat, a monkey, a horse, a dog, or a cat.


SEQUENCE LISTING (2 sequences)

SEQ ID NO: 1 - DNA, 21 bp, Artificial Sequence
  primer sequence
taaggcctacttcacaaagcg

SEQ ID NO: 2 - DNA, 23 bp, Artificial Sequence
  primer sequence
gcgattagaatgggtacaatgag